(19) United States
(12) Patent Application Publication
(10) Pub. No.: US 2014/0134124 A1
(43) Pub. Date: May 15, 2014

(54) SANITARY COMPOUND FOR USE ON SKIN

(71) Applicant: DONNA LYNN HUFF, Lancaster, CA (US)

(72) Inventor: DONNA LYNN HUFF, Lancaster, CA (US)

(21) Appl. No.: 14/077,096

(22) Filed: Nov. 11, 2013

Related U.S. Application Data
(60) Provisional application No. 61/725,868, filed on Nov. 13, 2012.

(51) Int. Cl. A61K 8/33; A61K 8/04; A61Q 19/10; A61K 8/39; A61K 8/49; A61K 8/97; A61K 8/92

(52) U.S. Cl. CPC A61K 8/33; A61K 8/97; A61K 8/046; A61K 8/922; A61K 8/39; A61K 8/498; A61Q 19/10
USPC 424/65

(57) ABSTRACT

A sanitary compound is configured to sanitize and condition human skin. The sanitary compound includes a mixture further comprising a first natural extract skin conditioner, a second natural extract skin conditioner, an amphoteric surfactant, a glycerol humectant, a chlorinated phenoxyl phenol odor control anti-bacterial agent, an ethoxylated sorbitan ester surfactant, a natural oil emollient, a tocopheryl acetate emollient, a fragrance, and an alcohol solvent. A preservative is mixed with the first solution, the second solution and water rendering the sanitary compound to be shelf stable. Applying the sanitary compound to the human skin sanitizes and conditions the human skin.

### RELATED APPLICATION

This application claims priority to provisional patent application U.S. Ser. No. 61/725,868 filed on Nov. 13, 2012, the entire contents of which is herein incorporated by reference.

### BACKGROUND

The embodiments herein relate generally to the use of a treatment composition to enhance skin health. Prior to embodiments of the disclosed invention, sanitary compounds were ineffective when used in sanitary wipes for sanitizing and conditioning human skin. Embodiments of the disclosed invention solve this problem.

### SUMMARY

A sanitary compound is configured to sanitize and condition human skin. The sanitary compound includes a mixture further comprising a first natural extract skin conditioner, a second natural extract skin conditioner, an amphoteric surfactant, a glycerol humectant, a chlorinated phenoxyl phenol odor control anti-bacterial agent, an ethoxylated sorbitan ester surfactant, a natural oil emollient, a tocopheryl acetate emollient, a fragrance, and an alcohol solvent. A preservative is mixed with the first solution, the second solution and water rendering the sanitary compound to be shelf stable. Applying the sanitary compound to the human skin sanitizes and conditions the human skin.

In some embodiments, the first natural extract skin conditioner can be a chamomile powder present in a quantity of 0.01-0.5% by weight. The second natural extract skin conditioner can be a solid aloe barbadensis leaf extract present in a quantity of 0.01-0.5% by weight. The amphoteric surfactant can be a disodium cocoamphodiacetate present in a quantity of approximately 1% by weight to ensure greater foaming. The chlorinated phenoxyl phenol odor control anti-bacterial agent is triclosan present in a quantity of 0.01-0.04% by weight. The natural oil emollient can be a wheat germ oil present in a quantity of 0.01-0.1% by weight. The tocopheryl acetate emollient can be present in a quantity of 0.01-0.1% by weight.

In some embodiments, the ethoxylated sorbitan ester surfactant is one of a Polysorbate-80 or a Polysorbate-20 that is present in a quantity of 1-4% by weight. The glycerol humectant is glycerin present in a quantity of 1-5% by weight. A weight ratio of the ethoxylated sorbitan ester surfactant to the glycerin is 4:3 for best emulsification.

In some embodiments, the preservative is polyaminopropyl biguanide present in a concentration of 0.04-0.3% by weight. In other embodiments, the preservative is at least one of a methylchloroisothiazolinone, a methylisothiazolinone, a DMDM hydantoin, and an iodopropynyl butylcarbamate.

### BRIEF DESCRIPTION OF THE FIGURES

The detailed description of some embodiments of the invention is made below with reference to the accompanying figures, wherein like numerals represent corresponding parts of the figures.

FIG. 1 shows a schematic view of one embodiment of the present invention.

FIG. 2 shows a schematic view of one embodiment of the present invention.

### DETAILED DESCRIPTION OF CERTAIN EMBODIMENTS

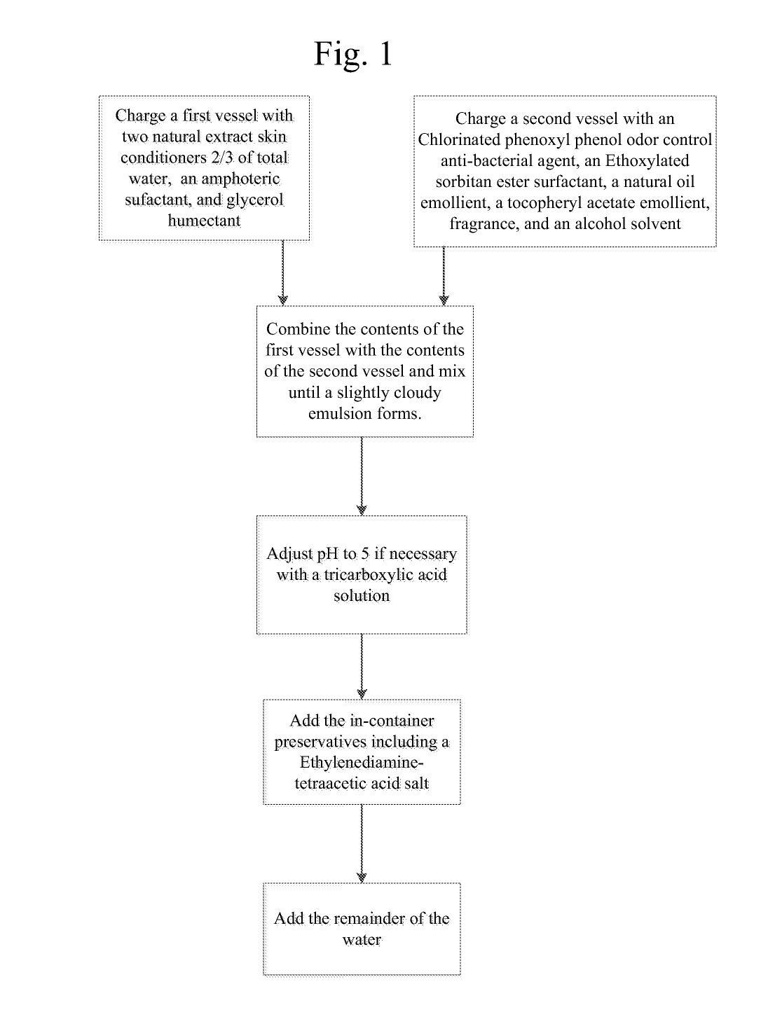

By way of example, and referring to FIG. 1, one embodiment of the sanitary compound comprises a first solution prepared in a first vessel combined with a second solution prepared in a second vessel. The first solution comprises a first natural extract skin conditioner, a second natural extract skin conditioner, a portion of the water necessary for the sanitary compound, an amphoteric surfactant, and a glycerol humectant. The second solution comprises a chlorinated phenoxyl phenol odor control anti-bacterial agent, an ethoxylated sorbitan ester surfactant, a natural oil emollient, a tocopheryl acetate emollient, a fragrance, and an alcohol solvent.

When the two solutions are mixed, a slightly cloudy emulsion should form. If not, then higher levels of the chlorinated phenoxyl phenol odor control anti-bacterial agent, the natural oil emollient, the tocopheryl acetate emollient, and the fragrance will yield cloudy emulsions. Lower levels of these ingredients yield a transparent mixture. The mixture, when shaken, should foam.

In the present use a slightly acid compound is desired. As used here, a slightly acid compound means a pH of about 5. If the sanitary compound is not slightly acidic, it can be brought to that point by adding an effective amount of a tricarboxylic acid.

Next, preservatives need to be added in order to ensure the shelf stability of the natural oil and extracts. The preservatives can be an isothiazolinone such as methylchloroisothiazolinone and methylisothiazolinone. The preservatives can also include a hydroxyalkyl hydantoin and an iodocarbamate such as DMDM hydantoin and iodopropynyl butylcarbamate. Another preservative that can be used is phenoxy ethanol. These preservatives can be used in any combination of one, two, three, or all four of these listed compounds.

In the United States, it is preferable to replace methylchloroisothiazolinone and methylisothiazolinone with polyaminopropyl biguanide to satisfy regulators. Likewise, for children under three years of age DMDM hydantoin and iodopropynyl butylcarbamate should also be replaced with polyaminopropyl biguanide.

The preservative can be added to a chelator such as ethylenediamine-tetraacetic acid (EDTA) salt. In some embodiments, disodium EDTA has been shown to be effective.

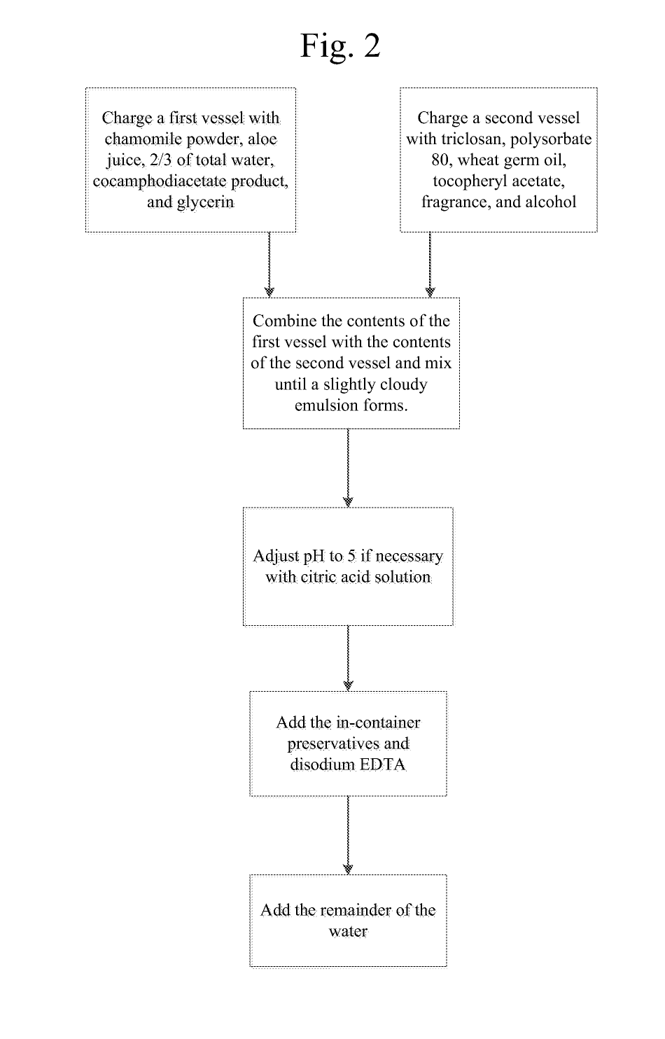

FIG. 2 shows the sanitary compound with more detailed substances identified. In this embodiment, the first natural extract skin conditioner is chamomilla recutita (matricaria) flower extract commonly called chamomile powder. The second natural extract skin conditioner is aloe barbadensis leaf juice though solid aloe barbadensis leaf extract would also be effective. The amphoteric surfactant is disodium cocoamphodiacetate, and a glycerol humectant.

Turning to the second solution, the chlorinated phenoxyl phenol odor control anti-bacterial agent is triclosan. The ethoxylated sorbitan ester surfactant is Polysorbate-80, though Polysorbate-20 can also be effective. The natural oil emollient is wheat germ oil. The tocopheryl acetate emollient is Tocopheryl acetate. The fragrance is parfum. The alcohol solvent is alcohol.

### Example 1

In this embodiment, water is present at 76.7% by weight. Alcohol is 14.7% by weight. Glycerin is 4.6% by weight. Polysorbate-80 is 3.4% by weight. Chamomilla recutita (matricaria) flower extract is 0.04% by weight as an active solid. Disodium cocoamphodiacetate is 0.24% by weight as an active surfactant. Aloe juice is present at 0.04% as an active ingredient. Polyaminopropyl biguanide is present at 0.1% as an active ingredient. Phenoxy ethanol is present at 0.5%. Citric acid is added as needed, typically in trace amounts, to adjust pH as noted above. Tocopheryl acetate is present at 0.01% by weight. Disodium EDTA is present at 0.15% as an active ingredient. Parfum was added as needed for fragrance depending on user preference.

In this embodiment, lower concentrations of triclosan, wheat germ oil, tocopheryl acetate, and fragrance resulted in a transparent mixture. The mixture is then embedded into a wipe and ready for personal use.

### Example 2

In these experiments the quantity of water was varied from 70-93% by weight, the quantity of alcohol was varied from 0-20% by weight, the quantity of glycerol was varied from 1-5% by weight and the quantity of Polysorbate-80 was varied from 1-4% by weight. The active ingredients were otherwise unchanged as noted in the first example.

A low range of water concentration can be offset by a higher range of alcohol concentration. More water yields a lighter product that has minimal residue. However, a higher alcohol range provides for a fast drying product. Zero alcohol can be used for a volatile organic compound (VOC)-free alcohol-free product.

A low range for glycerol and polysorbate results in a lighter product with minimum residue whereas, a high range for glycerol and polysorbate results in increased humectant properties. Experiments show that maintaining approximately 4:3 weight ratio of glycerol to polysorbate provides the best emulsification.

### Example 3

In these experiments various ranges were tried to find effective ranges of various substances in the sanitary compound. Chamomilla recutita (matricaria) flower extract was effective in a range of 0.01-0.5% by weight. Disodium cocoamphodiacetate was effective in a range of 0.1-1% as active surfactant with higher ranges resulting in more foaming. Aloe barbadensis leaf extract was effective in a range of 0.01-0.05% by weight. Wheat germ oil was effective in a range of 0.01-0.1% by weight. Triclosan was effective in a range of 0.01-0.04% by weight. Tocopheryl acetate was effective in a range of 0.01-0.1% by weight. Disodium EDTA was effective in in a range of 0.1-0.2% by weight.

### Example 4

In these experiments various ranges were tried to find effective ranges of various preservatives in the sanitary compound. Polyaminopropyl biguanide was effective in a range of 0.04-0.3% by weight. Methylchloroisothiazolinone and methylisothiazolinone were effective in a range of 0-0.0015% by weight. DMDM hydantoin was effective in a range of 0-1% by weight. Iodopropynyl butylcarbamate was effective in a range of 0-0.1% by weight.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A sanitary compound configured to sanitize and condition human skin; the sanitary compound comprising:
a mixture further comprising a first natural extract skin conditioner, a second natural extract skin conditioner, an amphoteric surfactant, a glycerol humectant, a chlorinated phenoxyl phenol odor control anti-bacterial agent, an ethoxylated sorbitan ester surfactant, a natural oil emollient, a tocopheryl acetate emollient, a fragrance, and an alcohol solvent;
a preservative mixed with the mixture and water rendering the sanitary compound to be shelf stable;
wherein applying the sanitary compound to the human skin sanitizes and conditions the human skin.

2. The sanitary compound of claim 1,
wherein the first natural extract skin conditioner is chamomile powder present in a quantity of 0.01-0.5% by weight; and
wherein the second natural extract skin conditioner is a solid aloe barbadensis leaf extract present in a quantity of 0.01-0.5% by weight.

3. The sanitary compound of claim 1,
wherein the amphoteric surfactant is disodium cocoamphodiacetate present in a quantity of approximately 1% by weight to ensure greater foaming;
wherein the chlorinated phenoxyl phenol odor control anti-bacterial agent is triclosan present in a quantity of 0.01-0.04% by weight.

4. The sanitary compound of claim 1,
wherein the natural oil emollient is wheat germ oil present in a quantity of 0.01-0.1% by weight; and
wherein the tocopheryl acetate emollient is present in a quantity of 0.01-0.1% by weight.

5. The sanitary compound of claim 1,
wherein the ethoxylated sorbitan ester surfactant is one of a Polysorbate-80 or a Polysorbate-20 that is present in a quantity of 1-4% by weight;
wherein the glycerol humectant is glycerin present in a quantity of 1-5% by weight; and
wherein a weight ratio of the ethoxylated sorbitan ester surfactant to the glycerin is 4:3 for best emulsification.

6. The sanitary compound of claim 1, wherein the preservative is polyaminopropyl biguanide present in a concentration of 0.04-0.3% by weight.

7. The sanitary compound of claim 1, wherein the preservative is at least one of a methylchloroisothiazolinone, a methylisothiazolinone, a DMDM hydantoin, and an iodopropynyl butylcarbamate.

8. A process for making a sanitary compound useful for sanitizing and conditioning human skin; the process comprising:
mixing a first natural extract skin conditioner, a second natural extract skin conditioner, an amphoteric surfactant and a glycerol humectant with a chlorinated phenoxyl phenol odor control anti-bacterial agent, an ethoxylated sorbitan ester surfactant, a natural oil emollient, a tocopheryl acetate emollient, a fragrance, and an alcohol solvent until a slightly cloudy emulsion forms;
adjusting the pH of the mixture to 5 by adding a tricarboxylic acid solution;
adding preservatives to the mixture;
adding water to the mixture

9. The process of claim 1,
wherein the ethoxylated sorbitan ester surfactant is one of a Polysorbate-80 or a Polysorbate-20 that is present in a quantity of 1-4% by weight;
wherein the glycerol humectant is glycerin present in a quantity of 1-5% by weight; and
wherein a weight ratio of the ethoxylated sorbitan ester surfactant to the glycerin is 4:3 for best emulsification.

10. The sanitary compound of claim 1, wherein the amphoteric surfactant is disodium cocoamphodiacetate present in a quantity of approximately 1% by weight to ensure greater foaming.
